(19) United States
(12) Patent Application Publication
(10) Pub. No.: US 2013/0053938 A1
(43) Pub. Date: Feb. 28, 2013

(54) SURFACE PROTECTIVE AND RELEASE MATRICES

(71) Applicants: Tiffany E. Miller, Shreve, OH (US); Daniel M. Storey, Longmont, CO (US); Barbara S. Kitchell, Holmes Beach, FL (US)

(72) Inventors: Tiffany E. Miller, Shreve, OH (US); Daniel M. Storey, Longmont, CO (US); Barbara S. Kitchell, Holmes Beach, FL (US)

(73) Assignee: METASCAPE LLC, Maple Grove, MN (US)

(21) Appl. No.: 13/564,088

(22) Filed: Aug. 1, 2012

Related U.S. Application Data
(63) Continuation-in-part of application No. 12/863,003, filed on Sep. 2, 2010, now abandoned.
Application No. 12/863,003 is a continuation-in-part of application No. 12/150,298, filed on Apr. 25, 2008, now abandoned.
(60) Provisional application No. 61/011,551, filed on Jan. 18, 2008.
(60) Provisional application No. 61/072,981, filed on Apr. 4, 2008.
(60) Provisional application No. 61/080,082, filed on Jul. 11, 2008.
(60) Provisional application No. 61/011,551, filed on Jan. 18, 2008.

(51) Int. Cl. C23C 16/50; B32B 3/30; A61K 35/12; A61F 2/10; A61F 2/82; A61K 9/00; B32B 3/26; B32B 1/02

(52) U.S. Cl. 623/1.1; 427/576; 428/312.8; 428/141; 428/35.2; 623/15.12; 424/424; 424/93.7

(57) ABSTRACT

A molecular plasma deposition (MPD) method in combination with an atomic layer deposition (ALD) procedure is used to produce amorphous, nonconformal thin metal film coatings on a variety of substrates. The films are porous, mesh-like lattices with imperfections such as pinholes and pores, which are useful as scaffolds for cell attachment, controlled release of bioactive agents and protective coatings.

### BACKGROUND OF THE INVENTION

1. Field of the Invention

The invention relates to extremely thin engineered nanoporous, nonconformal, amorphous metal or oxide coatings or films on a substrate surface. The surface deposited nanoporous metals can be used for controlled release of active agents, protective coatings, or scaffolds for cell adhesion.

2. Description of Background Art

Vapor and plasma based deposition of materials onto substrate surfaces are receiving increasing attention, in part because of the potential to create new surface features with desirable attachment, protective or time release properties. Nontoxic thin film surface coatings, for example, are of particular interest for implantable medical devices, where inflammation and fibrous encapsulation formation may cause significant problems. While nanostructured coatings would seem desirable for surgical implant purposes, highly adherent titanium porous coatings prepared with titanium sponge powders lack the appropriate roughness and/or porosity necessary for implants, and other coating techniques generally have been used to produce smooth, nonrough surfaces for nonmedical applications.

Vacuum arc deposition of metal oxides can be controlled to some extent to provide structured surfaces. One type of vapor deposition, chemical vapor deposition, is a chemical process in which one or more volatilized precursors are used to produce a highly conformal and pure thin film on a surface, primarily for applications in the electronic industry.

A related process, known as atomic layer deposition (ALD) utilizes alternating precursor exposure steps to produce conformal, non-porous thin films in a layer by layer fashion. ALD can be used to coat complex shapes with conformal material of high quality and is typically used to create smooth thin surfaces on microelectronic devices or to hermetically seal electronic equipment. A comprehensive review of the surface chemistry of the ALD process, reaction kinetics, growth mode and effect of reactions conditions is fully discussed in a recent review (Pruurunen, J. Applied Physics 97, 121301, 1-52, (2005).

ALD has been used to deposit ultrathin films on silica nanoparticles (Hakim, et al., 2005). The coatings exhibit complete coverage, uniformity and extreme conformality, which are considered the goal and advantages of using ALD for thin films. As practiced conventionally, the self-limiting growth mechanism of the precursors is determined by chemisorption on the surface. Where flux is large enough, the chemisorption layer is saturated. The excess precursor can be purged and conformality and trench filling occur. The resulting surfaces are smooth without nanorough features and highly conformal.

In efforts to increase bioactivity of implant device surfaces, atomic layer deposition (ALD) has been used to coat titanium or silicon substrates with a thin layer of crystalline titania. Bioactive anatase titania layers can be built up on the substrates and form hydroxyapatite when immersed in phosphate buffered saline (PBS) for several days in vitro. A disadvantage of the coatings is lack of adhesion of the hydroxyapatite to the ALD deposited smooth, conformal surfaces. For some applications, titanium thin films deposited from a radiofrequency generated plasma can be micropatterned onto quartz substrates. These surfaces reduce the generation of reactive oxygen species.

ALD techniques have been employed to surface engineer already nanostructured surfaces. Various nanotubular surfaces can be conformally coated using titania; for example, titania can be deposited onto a nanostructured surface so that all accessible surfaces are covered. Conformal surface coatings for underlying nano materials, including nanowires, nanolaminates and nanopore materials are known.

Surface microstructure is an important consideration in the design of many types of devices, including defined purpose implants. Surfaces serve many purposes and functions; for example, smooth biocompatible internal surfaces on coronary artery stents tend to discourage cell adhesion, which can inhibit or block blood flow. On the other hand, irregular surfaces modified by physical means such as etching or nanostructuring, have been investigated as scaffolds for attracting different cell types, which is a desirable function for bone implants where new bond growth is important in the healing process.

Medical devices are typically fabricated from polymers or metals, although only some materials are sufficiently biocompatible to be used as implants in orthopedic or surgical applications. Titanium is relatively unique because it shows little bio-incompatibility, due at least in part to formation of an oxide layer (i.e., titanium dioxide) on its outer surface.

Stents are small mesh-like matrices in a tube form placed in a blood vessel to maintain patency; i.e., to hold the vessel open so blood flow is not blocked. Coronary artery stents are typically metal, or a metal mesh framework, which over the years have been used extensively in heart patients. Unfortunately, bare metal stents are foreign to the body and tend to cause an immune response. The stent itself may induce rapid cell proliferation over its surface leading to scar tissue formation.

Coronary artery stents are typically composed of metal such as NiTiNOL or stainless steel and many have been developed with various coatings on the stent surface. The coatings not only protect the body from exposure to the metal but are also designed to release various drugs intended to inhibit or at least delay reclosing of the blood vessel in which the stent was placed. Multilayer coatings can be used, with one or more layers containing a drug or therapeutic agent, although thick coatings may lead to sloughing or provide foci for restenosis from surface cracks or other imperfections. Drug eluting layers when used in coronary stents most frequently contain immunosuppressive compounds, although anti-thrombogenic agents, anti-cancer agents and anti-stenosis drugs have also been used. Well-known and studied immunosuppressive drugs include cyclosporin A, rapamycin, daclizumab, demethomycin, and the like.

Protective coatings are used to modify surfaces in order to protect the underlying substrate; for example, stents have been coated with various polymer films, which not only add a protective layer to the base substrate but also act as a matrix or immobilization scaffold for different types of drugs. Protective films are often used to coat a metal matrix, which is typically stainless steel or titanium. The films themselves may serve dual functions as a protective hydrophilic surface and as a time-release matrix for therapeutic drugs.

Long term success of certain types of implants depends on surface characteristics for cell adherence and growth. Hip implants benefit from having a surface compatible with adherence and growth of osteoblast cells, while dental implants depend on soft tissue fibroblast adhesion and generation. This contrasts with the need for internal surfaces of stents and catheters to remain free of cell buildup so that those surfaces are generally smooth and lack structural defects that can act as foci for cell aggregation and adhesion.

Numerous methods and procedures for coating medical devices have been investigated and tested. Coatings have been applied using spin coat techniques, dipping, plasma deposition, surface flooding and the like. Recently, Heinrichs, et al., Key Engineering Materials, v. 361-3; 689-692 (2008) used atomic layer deposition (ALD) at 300° C. to deposit a thin crystalline TiO₂ coating on silicon, titanium 1 or titanium 2. When soaked in phosphate buffered saline, the polycrystalline epitaxially deposited films developed a layer of hydroxyapatite (HA). Unfortunately, lack of adhesion of the HA was observed with both the titanium and silicon substrates.

Elution of bioactive agents from implanted and indwelling medical devices has particular importance in the development of effective methods for administering therapeutics. Control of drug elution may be key to success in stents and other indwelling medical devices, which ideally should be able to remain in the body for long periods after implantation without restenosis.

A biocompatible coating with improved biocompatibility has been described using one or multiple layers of the natural product zein over or combined with taxol on substrate surfaces. Taxol exhibits a release profile that appears to be improved over PLA or PTX coatings.

Drug eluting stents have shown marked improvements in preventing the blood clots associated with stent thrombosis or “target lesion revascularization”. Two models of drug eluting stents are currently used. The CYPHER stent (Cordis) releases rapamycin, which has both immunosuppressive and antiproliferative properties. It is sold under the name Sirolimus and is used primarily as an immunosuppressive drug to prevent organ transplant rejection. The drug is produced by Streptomyces hygroscopicus and has the effect of blocking certain stages in the cell cycle G----S transition. The CYPHER stent is fabricated from stainless steel and is coated with a polymer that acts as a time-release carrier for the drug rapamycin.

The TAXUS™ Stent (Boston Scientific, Boston, Mass.) releases paclitaxel, which, like rapamycin, is an antiproliferative drug used primarily in cancer therapies. Paclitaxel interacts with microtubules so that the cell cannot undergo mitosis. The TAXUS™ Stent also utilizes a polymer drug carrier coated over a stainless steel substrate.

The benefits of drug-eluting stents are well recognized. Widespread use of these stents has resulted in significantly reducing restenosis of coronary arteries, which in the past was prevalent after coronary artery bypass graft surgery particularly with the use of bare metal stents. Nevertheless, stents fabricated from new materials or in new configurations (e.g., open scaffolding), would be desirable as drug carriers or matrices to improve drug efficacy or to act as carriers for newly developed drugs. Magnesium alloy stents, for example, may have some advantage over stainless steel stents; however this material has so far been tested only in animals.

The drugs selected for use as drug-releasing coatings are often imbedded in or associated with a polymer matrix, which is co-coated on the stent surface. Commonly used polymers, for example, are polyester lactides, polyvinyl alcohol, and cellulose. One example is a metal stent coated with a tripolide dispersed within a polymer matrix. Another example is an intravascular stent with a drug releasing coating composed of an immunosuppressive agent in a poly-dl-lactide polymer with a micro thick polymer undercoating on the stent. Polylactide polymers have also been used to prepare macrocyclic triene immunosuppressive coatings over a polymer underlayer. Not all polymers are biocompatible and some will not effectively coat the metals commonly used for fabricating stents and other medical implants.

Stent design has also been investigated, including various shapes for improved coating adherence and drug delivery. Development of more flexible materials such as metal mesh has improved stent function and in vivo adaptability. Stent structures that can be coated with varying thicknesses in different segments of the stent have been designed.

Despite the many improvements in stent design, materials and matrices for drug coatings, stents are subject to failure, due to development of inflammation at the implantation site or more commonly to restenosis of the artery. Metals such as tantalum and cobalt alloy based stents are under investigation as bare metal stents, although current thinking is that drug eluting stents are preferable because they minimize re-blockage in artery linings to a greater extent than bare-metal stents, particularly when used for FDA approved situations; i.e., “on-label”.

### SUMMARY OF THE INVENTION

The invention is the creation of a surface coating that exhibits a number of surface features which in combination are not produced from processes conventionally employed for atomic layer deposition (ALD) or atomic plasma deposition (APD). The coatings are thin, comparable to those typically produced by ALD or APD, but are nonconformal in contrast to ALD coatings which are typically highly conformal. The described coatings are nanostructured and porous, which are additional features of the coating which are not obtainable using plasma deposition processes as conventionally practiced.

The described method combines a modified form of APD with the serial deposition process of ALD and can produce nonconformal coatings on virtually any shape surface. In contrast to ALD coatings, the coatings as deposited do not follow the shape or profile of the substrate surface. The coatings can vary in thickness as measured from the coating surface to the substrate surface, yet remain nonconformal with respect to adherence to the substrate surface.

The new ALD/modified APD method produces coatings or films that are thin, adherent to the substrate, nanoporous and nonconformal with respect to the substrate. This method combines process steps typically used in molecular plasma depositions and serial deposition techniques used in atomic layer depositions (ALD).

ALD is a chemical vapor deposition process where source vapors are pulsed alternately into a reactor one at a time with non-deposited vapors separated by purging, usually by evacuation. Monolayers of deposited material deposited during each cycle can be built up to layers of desired thickness. Each time the precursors are introduced into the reaction chamber, the substrate surface is saturated and a self-limiting chemical reaction occurs to the extent of covering the entire surface.

The self-limiting growth mechanism of ALD provides several advantages including excellent conformality and capability to prepare nanolaminates in a continuous cyclic process.

Atomic plasma deposition (APD) utilizes a cathodic arc discharge to create a highly energized plasma generated from a metal target (the cathode). The particulates generated in the arc range in size from a few atoms to particulates in the micron range, depending on distance of the substrate from the arc, arc speed, voltage, etc.

APD is not conventionally used for plasma deposition of nonmetal or organometallic compounds, although such compounds can be deposited from plasma using a molecular plasma deposition (MPD) method. MPD is similar to APD except that the deposited plasma is not generated from the target. Atomized or colloidal suspensions of organometallic compounds are ejected from a conductive point source to form a corona discharge. The ionized plasma is directed into an evacuated chamber so that the ionized molecular plasma is deposited onto a substrate in an evacuated chamber. A typical apparatus includes a vacuum chamber with a small aperture, and a small bore, metallic needle connected to a tube connected to a reservoir holding a liquid suspension or solution of the material desired to be deposited. The reservoir holding the liquid is at atmospheric pressure. A power supply with the ability to supply up to 60 kV can be employed; however, the voltage attached to the needle is typically −5000 volts to +5000 volts. A substrate inside the vacuum chamber is centered on the aperture with a bias from −60 kV through 60 kV, including ground. A typical apparatus is shown in FIG. 12 and in FIG. 13.

The MPD process is described in U.S. Pat. No. 7,250,195, incorporated herein by reference. The deposited films are formed from ionized particulates and, like conventional APD films, are nanorough and surface adherent.

The described process is a modified combination of ALD and MPD. Organometallic precursors are introduced serially into an evacuated chamber. In contrast to ALD, a plasma is formed from the volatilized precursors under conditions similar to MPD procedures. The chemical reaction of the reactants on the substrate surface with purging of the unreacted materials over a series of cycles follows the course of reaction observed in ALD processes. Use of this modified ALD/MPD method produces a highly adherent film that is not conformal and exhibits a network of pore-like holes in a nanorough surface. None of the ALD, MPD or APD methods individually and conventionally used will provide films with this combination of characteristics. The coatings and films formed by the new process are highly adherent to the substrate surface and are excellent coatings for controlled release of bioactive agents from substrate surfaces due to the web-like pinhole surfaces. This is particularly noteworthy because ALD films are highly conformal and smooth and do not have the advantages of porosity or surface nanoroughness.

While processes other than ALD have been used to obtain coating films with one or more properties of the described coatings, none of APD, ALD or MPD has produced thin, nonconformal, nanorough, porous surface films. For example, ultrathin nanoporous titania films are described by Huang, et al. (2002). The Huang films were prepared from sol-gels by spin or dip coating followed by oxygen plasma treatment. The initially deposited composite films had flat, uniform surfaces which became roughened (nanostructured) only after oxygen plasma treatment.

Thin films have also been prepared by vacuum deposition. Guerra (U.S. Pat. No. 7,485,799) describes a thin titania film grown onto an irregularly shaped template. When the deposited film is subjected to stress, the original conformal coating delaminates briefly in certain spots after application of thermal or optical stress. The films prepared by the modified ALD/MPD method are nonconformal as deposited, yet strongly adherent to the substrate surface and do not readily delaminate.

The titania films described by LeClere, et al. (2008) are deposited by sputtering onto a substrate to provide a porous anodic film. The initially deposited film does not have porosity but develops irregularly shaped pores after anodization. The sputtered films are not uniformly deposited onto the substrate so that the top surface of the film is irregular.

In distinction to the known methods of film deposition, the described method is based on using a modification of chemical plasma and vacuum arc techniques to create nonconformal, porous, nanostructured metal films on a wide range of substrates. The new method produces films with mesh-like lattices punctured with pinhole and pore imperfections. FIG. 8 is an AFM (atomic force micrograph) photograph showing the irregular nano-sized surface pores. The surfaces are useful as scaffolds for cell attachment, controlled release of bioactive agents and as protective coatings.

As discussed, conventionally practiced ALD produces thin, smooth conformal surface films by deposition of vaporized precursors which react in sequential self-limiting surface chemical reactions to deposit films with high conformality. Under typical conditions, the precursors react only after adsorbing to the substrate surface. Excess reactant(s) are purged from the reaction chamber. By comparison, the described ALD/MPD method provides nanorough, porous nonconformal surface films using conditions similar to MPD but also using a cyclic sequential introduction and purging of precursors, as typically used in ALD procedures. The described surfaces are nanoporous, nonconformal, thin films.

The present invention discloses a method for obtaining nonconformal nanostructured thin coatings. In practicing the invention, conditions are adjusted so that, in contrast to ALD manufacturing procedures, nonconformal, amorphous deposits on substrate surfaces are produced. The new method produces nonconformal films on metal, polymer, ceramic and silicon substrates. Unlike typical metal-based crystalline coatings for electronic devices, metal oxide surfaces are amorphous, nonconformal and porous with a typical net-like appearance, as shown in FIGS. 7 and 8 for alumina and titania films, respectively.

The porous thin films produced by the described method are ideal scaffolds for cell attachment due to the nanostructure features. Such porous surfaces are also ideal as matrices for drug or other bioactive molecules, with applications for in vivo time release applications. Controlled deposition steps and proper substrate selection allow preparation of films that attract many types of cells.

The described method is exemplified with production of nonconformal titania and alumina surface deposited films, but may be used to provide films from other metal and oxide precursors; e.g., SiO₂, CdS, B₂O₃, V₂O₅, HfO₂, ZrO₂, ZnO and Pd. Silver oxide is expected to provide advantages not only as a thin adherent coating but also as providing antimicrobial properties on elution from the underlying substrate surface. Metal oxide surfaces can be deposited from a variety of metal oxides, metals, or combinations of metals and/or metal oxides.

As discussed, the described films are distinguished from ALD (atomic layer deposition films) because the ALD/MPD deposited titania or alumina is neither crystalline nor uniform. The present invention demonstrates that the surface properties of the deposited films are different from ALD deposited films and are suitable for surface coatings on implant devices, scaffolds for cell adhesion and matrices for bioactive molecules and controlled release.

The present invention utilizes relatively low temperature deposition conditions to produce thin nanoporous surface growth, in contrast to other vapor depositions which are conducted at much higher temperatures. Porous, amorphous surface films are cyclically deposited in thin layers, best described as monolayers.

Amorphous titania films prepared using the described method strongly attract osteoblast, endothelial, gingival fibroblast and periodontal fibroblast cells. The cells exhibit both increased proliferation and adherence compared to substrates with smooth coatings prepared by conventional deposition methods. Increased cell adhesion is observed on amorphous, nanorough, nonconformal titania films on titanium substrates compared to adhesion on smooth titanium or other substrates such as polymers.

The titania films described herein have different properties from conventionally deposited thin crystalline TiO₂ films. Conventionally deposited ALD titania films are typically deposited as epitaxial TiO₂ thin films with directional hydroxyl groups as a conformal film on single crystalline silicon wafers or titanium 1 or 2. In contrast, the coatings demonstrated herein are noncrystalline, nonformal, nanoporous, yet adhere strongly to the underlying substrate. The titania or alumina coatings do not slough or peel, in contrast to the significant flaking of surface coatings such as hydroxyapatite formed on deposited anatase TiO₂ (Heinrichs, et al., 2008).

In one aspect, the invention is directed to thin, porous metal oxide surface films that serve as time variable release coatings. A controlled number of ALD/MPD deposited layers of a metal oxide, illustrated with titanium oxide, can be deposited over a biomolecule, such as a drug, using the new process. Thickness of the film can be adjusted to control elution rate of the underlying drug attached or adhered to a substrate surface.

Controlled drug release films are particularly suitable for drug-eluting stents. In a further aspect of the invention, atomic plasma deposited layers of a metal oxide can be applied over a drug attached or adhering to a stent surface. The deposition is on an atomic scale such that each deposition can be considered in effect as a monolayer. A greater number of deposited layers increasingly hinders elution of a surface-attached drug, thus allowing customization of time release.

An example of controlled release of a material deposited on a surface is illustrated with a model test drug on a cobalt chromium substrate. When not covered with a layer of titania, rapamycin will elute almost immediately. However, by applying a surface in accordance with the described method, the drug elution from the substrate or matrix is significantly reduced.

The number of deposited coatings over drug-coated substrates has a distinct effect on drug release. Bare metal substrates, on which drug is deposited, show relatively rapid elution. Multiple alumina or titania top coats slow elution initially by at least several hours. The number of cycled layers, or monolayers, appears to have a controlling effect, with 10 layers having little effect on normal elution, while an increasing number of layers, on the order of 100s, show a definite effect in slowing elution.

Additional control of drug elution can be obtained by attaching a drug to a nanoroughened underlying substrate surface before applying an elution-controlling porous top coat. Previous work has demonstrated that nanostructured substrate surfaces are formed when materials are deposited from high energy plasmas by nanoplasma deposition (NPD), where the deposited materials, e.g., titanium, are metals. Biomaterials, including drugs and proteins, can be efficiently deposited, becoming firmly attached to underlying nanosurfaced metal substrates. The nanorough surfaces may be particularly useful on implant surfaces which act as matrices for biomolecule loading. This nanorough surface is preferably less than 100 nm thick.

In another aspect of the invention, a substrate is overlaid with a biomolecule eluting surface constructed of two or three layers, which can be described as a biolayer and a porous top coating or, where there are three layers, a nanorough surface, a biolayer, and a porous top coating, respectively. The layers may be formed on any substrate material including metals, polymers or ceramics, and are ideal for use on materials commonly used for medical implants, which are typically stainless steel, titanium, chromium cobalt or any of a variety of ceramics or polymers.

Multiple biomolecule eluting surfaces can be utilized in order to achieve the desired elution profiles. The biolayer need not be limited to a single type of compound or biomolecule, nor do the compounds need to be bioactive. A molecular plasma deposition (MPD) procedure described in U.S. Pat. No. 7,250,195, allows deposition of molecules individually or simultaneously if more than one molecular species is desired.

Coated drug surfaces are of particular interest in view of the wide range of therapeutic agents available to address adverse interactions encountered with medical implants. Currently popular drugs for use in arterial stents, for example, include anti-thrombotic and immunosuppressive agents. Other specialized implants may benefit from anti-microbial agents or anti-inflammatory drug coatings.

A particularly advantageous feature of the described method is the ability to deposit a relatively thin biolayer underlying the barrier layer. Many stents are multicoated with a protective polymer layer (the barrier layer over the substrate) followed by one or more layers (the biolayer) of polymer-attached or imbedded drug. Such multilayers add thickness to the lumen of a coated stent, which may exacerbate sloughing and contribute to manufacturing cost and quality control. Thus the thin coatings over a biolayer can impart a distinct advantage for medical implants.

The top layer over multilayer biocoatings, for example, can be a deposited film of a metal oxide such as titania or alumina. As a top surface, deposited layers function to some extent as a protective layer, but mainly act as a time release control for the underlying bioactive molecules comprising the biolayer. A set number of depositions; i.e. monolayers, will control the amount of drug elution, such as rapamycin, from near 100% elution within 2 hr for untreated surfaces to a much slower release over a period of 12 hours with 150 APD deposited titania layers.

Underlying surfaces; i.e., the substrates to which biomolecules are attached or in contact with, can have distinct functions and features. A nanorough substrate surface, if used, can be a thin film of deposited material such as any of a number of metals, ranging from 1 to up to 100 nm thick, depending on desired substrate coverage and roughness. Coatings up to 500 nm may be useful, but generally thicker coatings or layers on the order of several hundred nm appear to be most practical for implant devices.

A biolayer on the substrate, whether nanorough or smooth, can be deposited to obtain a select coverage or activity. Biolayers may be any of several molecular types, including metals, proteins and many organic molecules. The procedure is described and exemplified in U.S. Pat. No. 7,250,195 (2007). The biolayer may also be applied using ink jet printing, spin coating, dip-coating and similar methods well-known in the art.

A top or final layer deposited as described herein can be used to form a porous surface and can be deposited to a thickness appropriate for a desired elution rate of one or more biomolecules. The overall top layer is thin, less than 1 nanometer to several hundred nanometers thick depending on the elution rate desired. Overall thickness of the substrate coatings (the biomolecule and the top surface material) and the types of biomolecule(s) will determine the elution rate.

An advantage of selecting titania as a top layer is titania's recognized compatibility in vivo and its track record of use in medical implants. Titania is nontoxic and is not associated with an immune response.

The base substrate can be selected from a metal, ceramic or polymer including copolymers, biocompatible polymers such as polylactic acid and dissolvable polymers, depending on intended use. For example, a biomolecule or other agent can be attached to or coated over gold or silicon in applications such as biosensors.

Typical substrate materials used in devices such as orthopedic implants, dental implants, catheters and indwelling permanent or long-term devices include metals and plastics. Stainless steel, titanium and cobalt chromium stents are of particular interest in view of widespread use in heart vessel replacements. An additional advantage of titania top layers, as discussed, is that release of bioactive materials from substrate surfaces can be tailored to the properties of the underlying biomolecule.

While titania and alumina are exemplary of metal oxides that can be deposited using the described method, other metals are expected to exhibit similar properties. Alumina films exhibit properties similar to titania. Other metals such as hathium, iridium, platinum, gold, and silver can be produced as thin surface films with analogous properties.

### BRIEF DESCRIPTION OF THE FIGURES

Except as otherwise noted, all coatings described in the figures were deposited by the modified ALD/MPD method described herein.

FIG. 1 illustrates the arrangement of a substrate (1) coated with a biomolecule (2) and overlaid with deposited titanium oxide (3) that allows elution of the biomolecule.

FIG. 2 illustrates a biomolecule-eluting system with repeating layers of biomolecule and coating on a substrate surface (1); layers of biomolecule (2a and 2b) overlaid with an APD titanium oxide film (3a and 3b) over each layer of drug.

FIG. 3 is a graph showing an elution profile for rapamycin deposited on a substrate covered with titania, in the arrangement illustrated in FIG. 1. X represents the control without the titanium oxide coating film over the rapamyxin; , ▴, ▪, and represent deposited titania surface films of thicknesses 25 nm, 50 nm and 75 nm, respectively.

FIG. 4 is a graph showing a rapamycin elution profile from the multilayer system illustrated in FIG. 2. X is the control with only the drug applied to the substrate; 0 corresponds to elution of the drug applied in two layers, the first layer 2a covered with a 30 nm thick titanium oxide film 3a and the second layer 2b covered with a 35 nm thick titanium oxide film 3b.

FIG. 5 is a graph showing osteoblast cell adhesion on amorphous titania coated on 316L stainless steel after 4 hr and 3 days incubation; SS1 is coated with amorphous titania about 5 nm thick; SS2 about 8 nm thick; SS3 about 12 nm thick and SS4 about 15 nm thick; C is stainless steel control without a titanium coating; *indicates standard deviation.

FIG. 6 is a graph showing endothelial cell adhesion on amorphous titania coated on titanium grade 1 after 3 days; Ti-1 coating is about 5 nm thick; Ti-2 coating is about 10 nm; thick; Ti-3 coating is about 20 nm thick; Ti-4 is about 40 nm thick; Ti-5 is about 60 nm thick; Ti-6 is about 80 nm thick; C is a control without the APD titanium surface coating; *indicates standard deviation.

FIG. 7 is an AFM image of deposited alumina on a silicon substrate.

FIG. 8 is an AFM image of deposited titania on a silicon substrate.

FIG. 9 is an AFM image of a typical smooth surface using conventional ALD methods to deposit a titania coating.

FIG. 10 is an AFM image of deposited titania showing the nanorough surface.

FIG. 11 shows several metal parts coated with deposited titania with surfaces varying from curved to flat.

FIG. 12 is a sketch of a molecular plasma deposition apparatus: vacuum chamber 1; high voltage power supply 2, substrate holder 3; substrate 4; high voltage power supply 5; needle 6; feeder tube to needle 7; orifice 15 into reservoir 8; colloidal liquid suspension 9.

FIG. 13 is a sketch of a modification of the apparatus of FIG. 12: vacuum chamber 1; high voltage power supply 2; substrate holder 3; substrate 4; high voltage power supply 5; needle 6; feeder tube 7; orifice 15 into reservoir 8; liquid suspension 9; secondary chamber 10; secondary chamber gas supply 11; secondary chamber gas supply line 12; pressure regulator 13; gas line from regulator 14.

### DETAILED DESCRIPTION OF THE INVENTION

### Background

The present invention utilizes an atomic plasma deposition technique to produce nanoscale thickness films on surfaces. The films are produced using a modified molecular plasma deposition (MPD) technique to achieve surfaces ranging from sub-nanometer thicknesses up to hundreds of nanometers. Importantly, the process produces thin films that are amorphous rather than crystalline and which have a porous character, making them ideal for time-release applications, cell adhesion matrices and protective coatings.

The process as described is distinguishable from any of ALD, MLD, IPD, NPD and MPD plasma deposition procedures, as defined below.

### DEFINITIONS

Conformal and nonconformal are terms of the art referring to the conformity or lack of conformity of a coating or film with respect to the shape and profile of the underlying substrate. Conformal films follow the shape of the substrate surface exactly while nonconformal films do not completely take the shape of the surface on which they are deposited.

Atomic layer deposition (ALD) creates conformal, pinhole free thin films which are typically used in the electronics and solar cell industries. The process is defined by two sequential, self-limiting surface reactions.

Cathodic-anodic plasma discharge is a cathode/anode system of creating a plasma discharge at high (>100V DC, AC or RF).

Molecular Layer Deposition (MLD) is similar to ALD and is used to grow polymer films. Typically, two bifunctional monomers can be employed to obtain “layer by layer” deposition.

Ionic Plasma Deposition (IPD) is the vacuum deposition of ionized material generated in a plasma. The plasma is produced by applying high voltage to a cathode target. The ionized plasma particles so generated are deposited on a substrate which acts as an anode.

Nanoplasma deposition (NPD) utilizes an ionized gas produced by a DC current in order to deposit the ionized species onto a selected substrate surface. The thickness of films and coatings produced in this manner can be controlled but deposition is not uniform. This results in a nanorough surface.

MPD or molecular plasma deposition also utilizes a plasma, but produces the plasma from solutions or suspensions of materials introduced between the high voltage cathode and substrate anode, set up in a manner similar to IPD.

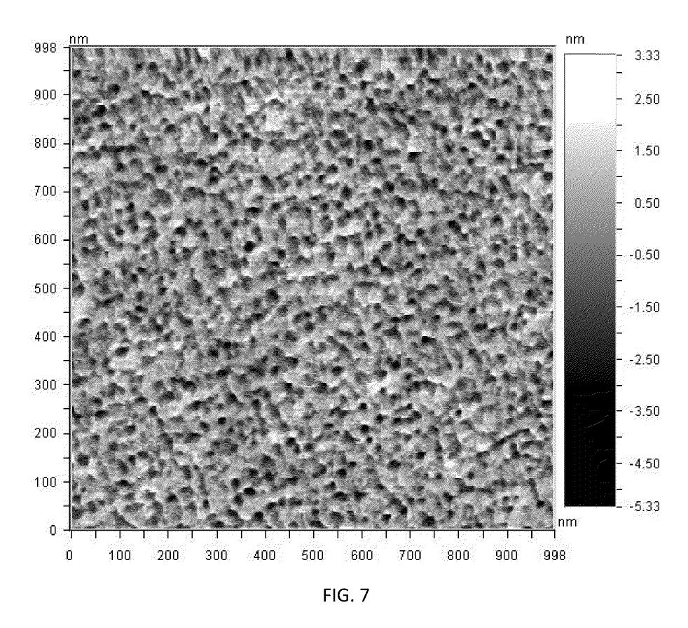

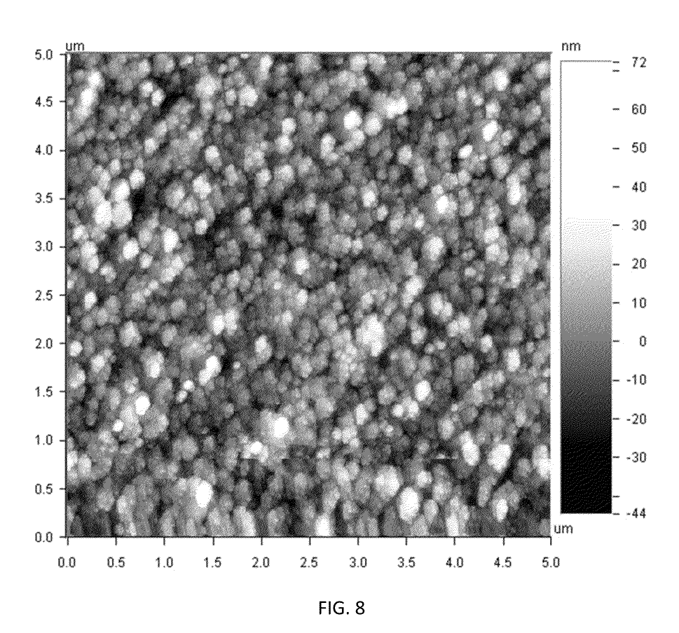

The modified ALD/MPD method of the present invention is performed under sub-optimal conditions such that deposited films are grown via two sequential reactions resulting in porous or mesh-like surfaces. Pinhole and pore imperfections in these surfaces are shown in FIG. 7 and FIG. 8. The films are amorphous and do not conform to the substrate surface, instead forming an adherent, yet nanorough, surface as deposition occurs.

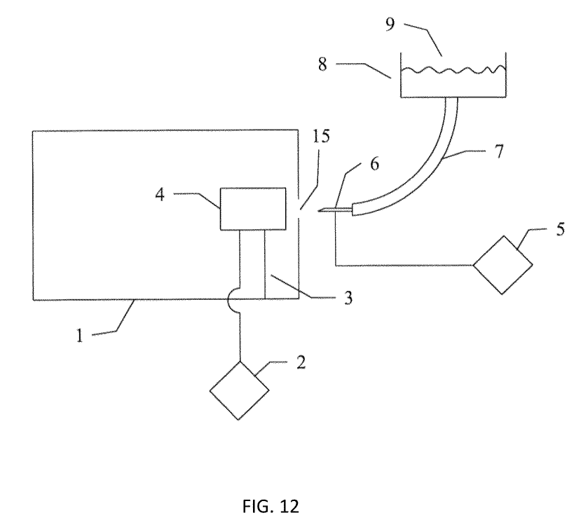

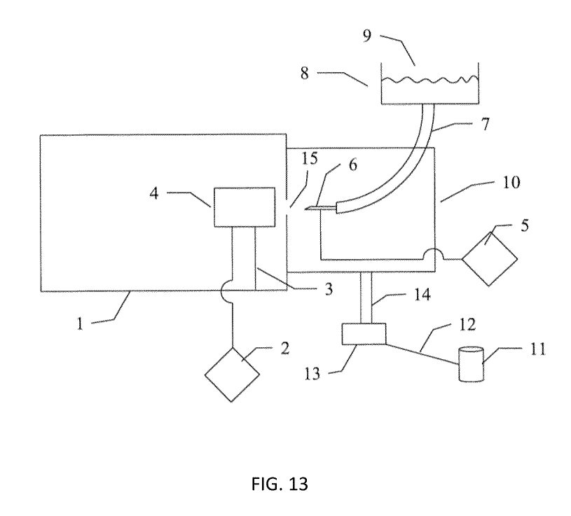

The modified ALD/MPD method is similar in some respects to ALD and MLD in using sequential depositions but utilizes a highly charged plasma deposition. FIGS. 12 and 13 illustrate a typical apparatus for generating the plasma from titania or alumina precursors which are then injected into a vacuum chamber housing the substrate. Imperfections such as pinholes and pores are incorporated into a thin film, thereby creating a mesh-like structure. Two sequential surface reactions are performed.

As discussed, the described method produces nanoscale thin films by utilizing a chemical process that sequentially promotes a chemical reaction on a substrate surface between an organometallic compound and an oxygen source. Nonconformal, porous surfaces are formed, using intermittent, cyclic deposition of nanolayers to form films ranging from sub-nanometer thicknesses up to hundreds of nanometers in thickness. Virtually any type of substrate can be selected; for example, stainless steel, titanium, titanium alloy, magnesium alloy, cobalt alloy, ceramics, silicon, glass or polymers, including biocompatible and dissolvable polymers.

Nanophase single or multiple layer time release coatings over drugs attached to metal surfaces are another aspect of the invention. The coatings are deposited over a drug attached to a porous metal substrate using an atomic plasma deposition procedure. Porosity of the substrate and the number of deposited layers control drug release when the attached drug is exposed to an aqueous medium.

The invention provides methods for preparing nanoporous surfaces over immobilized or otherwise attached molecules on an underlying surface. The deposited metal oxide serves to protect the underlying biomolecule but does not prevent elution of the biomolecule. Elution rate is determined by several factors in addition to the porosity of a thin film, including thickness of the film, the metal oxide used, species of biomolecule, and the nature and degree of biomolecule adherence to the underlying substrate. The fluid environment to which the coated material is exposed is also a factor. In most applications, it is desirable to use coatings over drugs appropriate for in vivo use, which are well characterized regarding activity and ability to attach to substrates.

The thickness of coatings can be readily controlled by cycling the deposition conditions. For an exemplary drug rapamycin, relatively thin layers in the range of 25 to about 75 nm thickness provide a range of elution profiles, indicating that it is a matter of routine to determine appropriate thickness of the porous topcoat, in this case titania, but it could also be other metals such as aluminum oxide, for a desired elution rate. It should be noted that surface film thickness is not the sole factor to consider in achieving a desired elution. Elution rates will necessarily depend on the chemical characteristics of the biomolecule and on the adhesion or binding of the biomolecule to the base substrate. The biomolecule can be covalently attached to some substrates; for example, a gold or other substrate with an activated surface. Substrates are not necessarily metal, and polymeric substrates could be combined with bioactive molecules

As discussed, biomolecule adhesion to the matrix or substrate is a factor in the elution characteristics. Several methods of attaching biomolecules to a surface are known, including spraying, dipping, ink jet printing, and deposition methods such as vacuum arc plasma deposition. Adherence or binding of the biomolecule may be affected by both the substrate material itself and surface roughness. Non-covalent interactions may be enhanced on nanorough surfaces. Surface area can be increased by mechanical means or by laser or plasma surface exposure. Deposition of a metal onto a substrate using NPD can be used to pickle the surface with micro or nanoparticulates, which generally increases adherence to these surfaces compared to smooth surfaces.

On the other hand, the disclosed titania surfaces are nanoporous and are appropriate as protective surfaces for mitigation of potential toxic effects from certain plastics or polymers that are in contact with the body or from potentially toxic drugs. A toxic agent can be controllably eluted from an indwelling probe or other device in such a manner that the toxic agent is targeted to a specific location, either by positioning of the device and/or because the material itself targets to a particular organ or type of cell; e.g., a targeting vector or antibody.

An additional advantage of the described titania surfaces is their extremely thin profiles, which are resistant to sloughing. This is not only economical but also, at least in the case of titania, provides a surface which consists of an inert material that is not known to be immunogenic and is nontoxic.

### EXAMPLES

The following examples are provided as illustrations of the invention and are in no way to be considered limiting. Temperature, cycling sequence, and similar precursors can be used within the contemplated scope of the invention.

Materials and Methods

Rapamycin was purchased from L.C. Laboratories (Woburn, Mass.) and used without further purification. Elution tests were performed in a 60% 1× phosphate buffered saline (PBS) and 40% methanol solution.

Osteoblasts (CRL11372) were purchased from American Type Culture Collection (ATCC); endothelial cells from VEC Technology (Rensselaer, N.Y.). All substrates were sterilized under UV light overnight prior to cell attachment measurements. Human osteoblasts, population numbers 5-7) in Dulbecco's Modified Eagle Medium (Gibco) supplemented with 10% fetal bovine serum (Hyclone) and 1% penicillin/streptomycin (Hyclone) were seeded at a density of 3500 cells/cm² onto the substrate and then incubated under standard cell culture conditions (humidified, CO₂/95% air environment, 34° C.). After 4 hr incubation, the substrates were rinsed in a phosphate buffered saline to remove any non-adherent cells. After rinsing, cells remaining attached to the substrate were fixed with formaldehyde (Aldrich), stained with Hoescht 33258 dye (Sigma), and counted under a fluorescence microscope (Leica, DM IRB). Five random fields were counted per substrate. All substrates were run in triplicate Standard t-tests were used to check statistical significance between means. Endothelial cells were incubated and counted in the same manner as the osteoblast cells.

The MTT [3-(4,5-dimethylthiazol-2-yl)-2,5-diphenyltetrazolium bromide] assay is based on the ability of a mitochrodrial dehydrogenase enzyme from viable cells to cleave tetrazolium rings of the pale yellow MTT and form dark blue formazan crystals which are largely impermeable to cell membranes, resulting in accumulation in healthy cells. Solubilization of the cells by addition of a detergent results in the liberation of the solubilized crystals. The number of surviving cells is directly proportional to the amount of formazan product produced. Color can be quantified by colorimetric assay and read on a multiwall scanning spectrophotometer (ELISA reader).

For 7 day tests, the MTT assay was used to quantify and check viability of the cells. A colorimetric procedure was employed to measure cell growth. Living cells reduced MTT which is yellow in color to formazan which is purple. The purple color was quantified using UV absorbance at 500-600 nm.

The cells on the surface of the substrates were measured and reported as cells/cm².

AFM images and surface roughness data were collected using a Park Systems XE-150 atomic force microscope operating in non-contact mode. Samples were affixed to metal mounting discs with carbon tape and an NSC16/AIBS cantilever was used for sampling. The scan sizes were 1×1 μm² areas. Data processing was performed with XE1 v1.6 software.

### Example 1

### ALD/MPD Deposited Thin Titania Films

Metal oxide films can be deposited on various substrates from organometallic precursors using a new combination of steps adapted from ALD and MPD methods. In a typical example, titanium oxide was plasma deposited in self-limiting reactions from a reaction chamber supplied with alternating exposures of volatilized 30% hydrogen peroxide (in water) and titanium isopropoxide (TIIP), using nitrogen as the carrier gas. The apparatus for generating the precursor molecular plasma is sketched in FIGS. 12 and 13. The charged molecular species are generated under atmospheric conditions at room temperature.

Before the deposition cycles were initiated, a pulsed plasma was run at 500 v, at 100 kHz for 30 min. to prepare the substrate surface. TIIP was injected into the system through the valve connected to a needle like apparatus electrically isolated from the system, but attached electrically to a high voltage (in this example, up to 1500 v, but can be DC, pulsed DC, which was used in this example, AC, or RF power supply negative terminal. The positive was hooked to the chamber and the parts onto which the coating was deposited. The H₂O₂ precursor was also hooked up to a separate power supply using the same boundary conditions and attachments. The two supplies were alternatively turned on and off with a pulsed DC mode. The pulsed plasma was continued through the entire deposition cycle. An exemplary apparatus is shown in FIG. 12.

More particularly, to produce and deposit a titanium oxide coating on a substrate surface, the following reaction sequence was used: evacuating the reaction chamber to 1×10⁻⁴ Torr; stopping the evacuation during a 0.2 sec introduction of hydrogen peroxide into the closed chamber, a 10 second delay during which the vacuum is released, closing of the chamber and a 0.2 second introduction of titanium isopropoxide, then initiating a 10 sec delay during which time the chamber is evacuated and the process repeated. The temperature of the reaction chamber was 160° C. Introduction of the volatilized precursors, TIIP and H₂O₂, into the reaction chamber was alternated for 1500 cycles, producing a film of about 120 nm in thickness. Deposited film thickness can be controlled by the number of cycles conducted. For titania films temperature of the chamber is generally at or below 165° C.

### Example 2

### ALD/MPD Deposited Thin Alumina films

In a second example, aluminum oxide was sequentially deposited in self-limiting reactions from the reaction chamber supplied with alternating exposures of volatilized trimethyl aluminum and water using nitrogen as a carrier gas. The same parameters used for producing a titania thin film from molecular plasma precursors as described were used to produce aluminum oxide and deposit the plasma as a thin alumina film.

The following reaction sequence was used: 0.2 sec exposure of water in the reaction chamber evacuated to 1×10⁻⁴ Torr, 10 sec delay, 0.2 sec exposure of trimethyl aluminum, and 10 sec delay. The temperature of the reaction chamber was 600° C. Introduction of volatilized precursors, water and trimethylaluminum, into the chamber was alternated for 1000 cycles, producing a film of about 90 nm in thickness. For alumina films, temperature of the chamber is preferably at or below 160° C.

### Example 3

### Titania Films on a Drug Coated Substrate

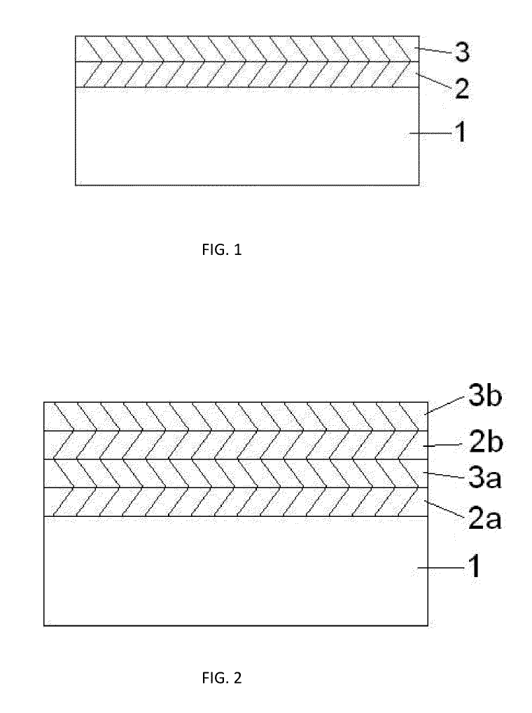

Using the APD/MPD method described in Example 1, titanium oxide thin films were grown over rapamycin previously deposited on a stainless steel substrate by the MPD method described in U.S. Pat. No. 7,250,195. The titania film was grown over the deposited rapamycin by sequential self-limiting reactions of titanium isopropoxide and an oxygen source. FIG. 1 is a schematic illustration of the relative thicknesses of the rapamycin coated substrate and the overlying surface formed from the deposited titania.

### Example 4

### Elution of Titania Coated Rapamycin

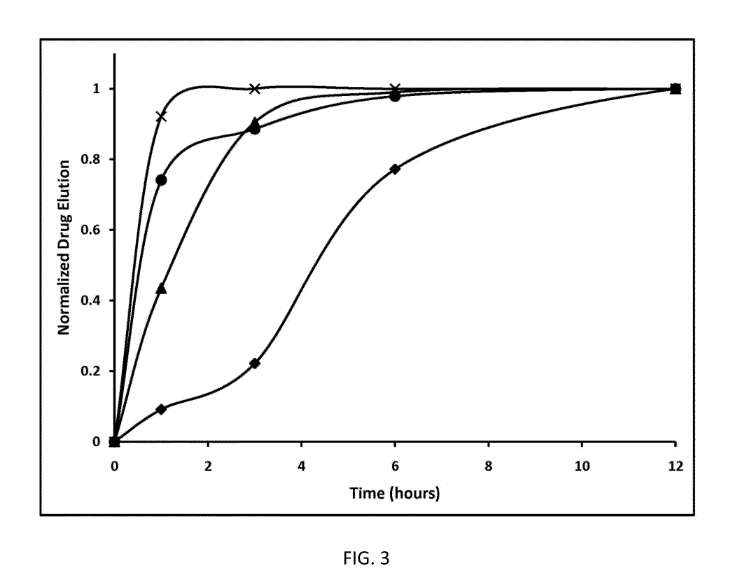

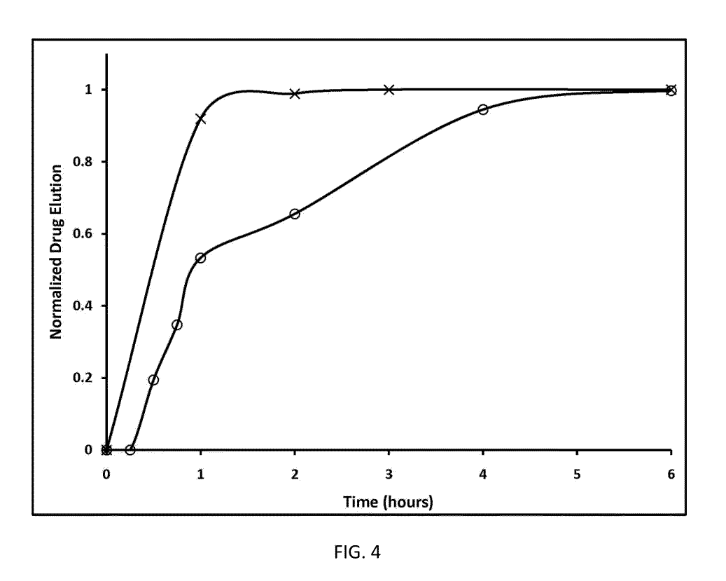

FIG. 3 shows the amount of rapamycin elution from deposited titania of various thickness normalized to the control without the titania. , ▴, ▪ represent ALD/MPD deposited titania surface films of thicknesses 25 nm, 50 nm and 75 nm respectively with respective release of the drug over up to about 6 hr for the 25 and 50 nm thick layers and up to about 12 hr for 75 nm thick top layer. The rate of drug release into a PBS/methanol solution is roughly proportional to the thickness of the surface deposited material, at least for layers up to about 100 nm thick.

### Example 5

### Titania Multilayer Films for Controlled Release

A titanium oxide film was deposited over rapamycin deposited onto a cobalt chromium substrate. Rapamycin was deposited from a colloidal solution using the plasma generation procedure described in Example 1. A coating of titanium oxide was deposited over the rapamycin using the ALD/MPD process described and as depicted in the cross section of FIG. 1.

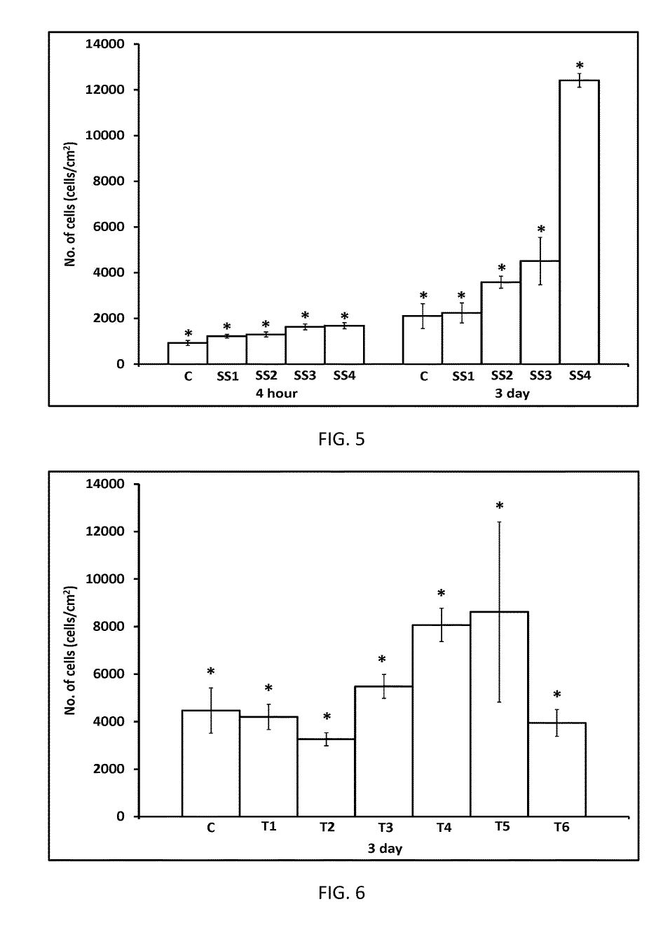

FIG. 6 is a rapamycin elution profile for release from a cobalt chromium substrate surface with deposited titania on two separate layers of deposited rapamycin as depicted in the cross section of FIG. 2. In this example, the thicknesses of the titania layers 3a and 3b were 30 and 35 nm respectively. The control (x) has no top coating and the drug releases almost completely within about 2 hours. With the multiple layer coatings, rapamycin is initially released fairly rapidly, but then slows significantly up to about 4.5 hr compared with the control.

### Example 6

### Cell Adhesion to ALD/MPD Deposited Titania Surfaces

Human osteoblast cells were incubated on amorphous titania films prepared as described in Example 1. Underlying substrates used were polycarbonate, stainless steel (316L), or titanium (grade 1 and grade 2). Thickness of the amorphous films ranged from 5 to 80 nm thicknesses. FIG. 5 is a graph showing osteoblast cell adhesion on amorphous titania coated on stainless steel after 4 hrs and 3 days incubation; SS1 (stainless steel) is coated with amorphous titania about 5 nm thick; SS2 substrate is coated about 8 nm thick; SS3 is coated about 12 nm thick and SS4 coated about 15 nm thick. C is a stainless steel control without a titanium coating; * indicated standard deviation.

Similar results are seen with endothelial cell adhesion on amorphous titania coated by the process of Example 1 on titanium grade 1 substrate, as shown in FIG. 6 comparing adhesion after 4 hr and again after 3 days incubation. Ti-1 substrate is coated with 5 nm thick amorphous titania; Ti-2 with 10 nm thickness; Ti-3 with about 20 nm thickness; Ti-4 with about 40 nm thickness; Ti-5 about 60 nm thick and Ti-6 is about 80 nm thick. C represents a stainless steel control without an ALD/MPD titanium coating; * indicated standard deviation.

Similar tests with gingival or periodontal ligament fibroblast cells on ALD/MPD amorphous titanium or stainless steel coated substrates showed similar results. Film thicknesses ranged from 5 to 80 nm.

ALD/MPD deposited titania films exhibited different cell attracting characteristics depending on the particular underlying substrate. Polycarbonate substrates coated with thin titania films exhibited at least an order of magnitude greater adhesion for osteoblast cells than for other types of cells such as gingival and periodontal fibroblasts.

### Example 7

### Characteristics of ALD/MPD Amorphous Nonconformal Thin Films

The new ALD/MPD method was used to deposit thin titania oxide films. The films exhibited mesh-like surfaces, see FIG. 8, with pinhole-size and pore imperfections. The amorphous titania films form a nanorough, porous surface as deposition occurs.

Images and surface roughness data were collected using a Park Systems XE-150 atomic force microscope (AFM) operating in non-contact mode, described in Materials and Methods.

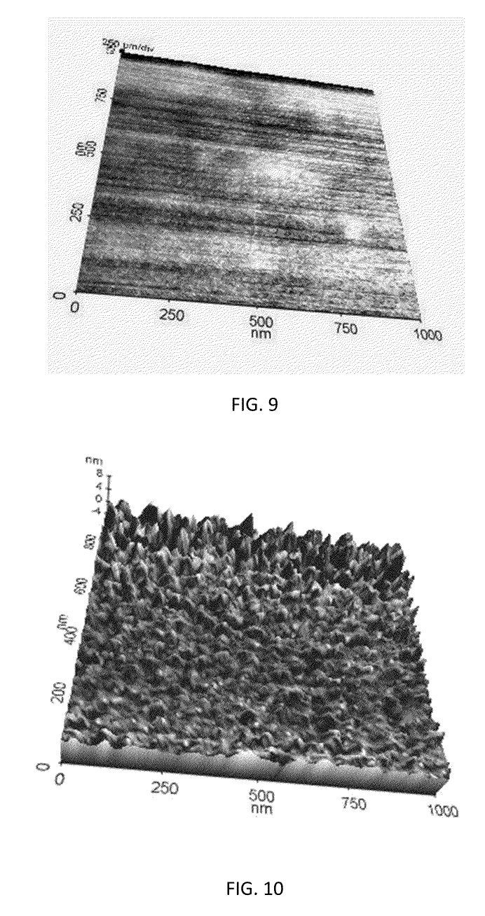

An AFM image of alumina on a silicon substrate is shown in FIG. 7 and of titania on a silicon substrate in FIG. 8. Both were deposited under the ALD/MPD conditions set forth in Examples 1 and 2. For comparison, FIG. 9 is an image of a smooth titania surface deposited under typical ALD plasma deposition conditions while FIG. 10 illustrates the nanorough surface of ALD/MPD deposited titania.

Titania coatings deposited under the conditions of Example 1 did not chip or flake and did not peel from the substrate when subjected to a standard tape test.

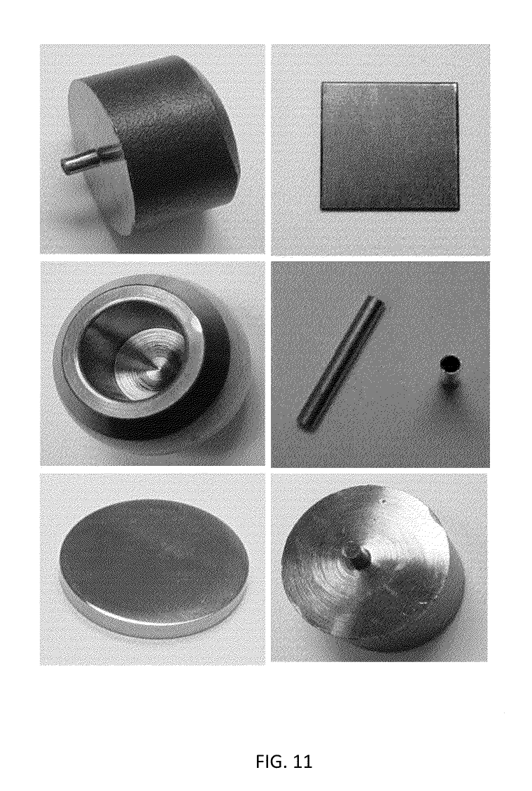

Several devices with different shapes were coated with titania under the described conditions. The coatings were uniform and of various thickness. FIG. 11 shows examples of differently shaped metal parts successfully coated with highly adherent titania ALD/MPD coatings. Color of the deposited films varied with coating thickness, as shown in Table 1 for thicknesses in the 1 nm-200 nm range. Color changes are not visually detectable until the deposited film is about 50 nm thick.

TABLE 1

| Thickness (nm) | Color |
|---|---|
| 30 | Substrate color |
| 50 | Golden yellow |
| 75 | Dark blue |
| 100 | Green blue |
| >100 | No change |

### Example 8

### Comparative Characteristics of Thin Films

Table 2 compares the typical film characteristics of coatings prepared by APD, MPD and ALD with the films deposited by the modified ALD/MPD deposition method described herein.

TABLE 2

| | | THICK- | | Crystal- |
|---|---|---|---|---|
| | SURFACE | NESS | CONFORMALITY | linity |
| APD | Nanostructured | Thin | Conformal or | Crystal |
| | Nanoporous | | nonconformal | |
| MPD | nanostructured | Thin | Nonconformal | Amorphous |
| ALD | Smooth | Thin | Conformal | Crystal |
| Modified | Pores/meshlike | Thin | Nonconformal | Amorphous |
| ALD/ | nanostructured | | | |
| MPD | | | | |

What is claimed is:

1. A method for preparing a nonconformal, porous, amorphous metal or metal oxide surface film, comprising:
(a) evacuating a heated reaction chamber housing a substrate maintained at an induced potential opposite from a conductive point source;
(b) introducing an oxygen source plasma into the evacuated chamber;
(c) releasing the vacuum;
(d) generating a molecular plasma under atmospheric conditions from a volatile metal based precursor ejected from the conductive point source having a high potential gradient;
(e) evacuating the chamber;
(f) directing the generated plasma through an orifice into the evacuated chamber; and
(g) repeating steps (a)-(f) a sufficient number of times for the oxygen source and metal precursor injected into the chamber to react on the substrate surface to form a nonconformal, porous, amorphous metal oxide surface film.

2. The method of claim 1 wherein the oxygen source is water or hydrogen peroxide.

3. The method of claim 1 wherein the surface film is formed to a thickness up to 500 nm.

4. The method of claim 1 wherein the substrate is a metal, polymer, silicon, ceramic metal, stainless steel, titanium, titanium alloy, magnesium alloy or cobalt alloy polycarbonate, polyvinyl chloride (PVC) or 316L stainless steel, titanium grade 1, titanium grade 2, a biodegradable or biocompatible polymer or copolymer.

5. The method of claim 1 wherein the metal-based precursor is titanium isopropoxide or trimethyl aluminum.

6. The method of claim 5 wherein the reaction chamber temperature is at or below 165° C. for formation of a titania film from a titanium isopropoxide precursor.

7. The method of claim 5 wherein the reaction chamber temperature is at or below about 20° C. to about 160° C. for formation of an alumina film from a trimethyl aluminum precursor.

8. The method of claim 5 wherein the reaction chamber temperature is at or below 165° C. for formation of a titania film from the titanium isopropoxide precursor.

9. An amorphous, porous, nonconformal, thin titania or alumina surface film produced by the method of claim 1.

10. The film of claim 9 which comprises the surface of a medical device.

11. The film of claim 9 which is a controlled release surface over a biomolecule attached to an underlying substrate, wherein the biomolecule elutes in a time-dependent manner.

12. A controlled release surface, comprising, a biomolecule deposited on or attached to a substrate surface;
a nonconformal, porous titania or alumina film deposited over the attached biomolecule by the method of claim 1; and
optionally multilayer deposited biomolecules each coated with said titania or alumina film
wherein the biomolecule elutes from the nonconformal, porous titanium or alumina film in a time-dependent manner.

13. A nanorough amorphous nonconforming deposited thin titania or alumina surface film about 100 nm to about 500 nm thick prepared by the method of claim 1 by introduction of each precursor in 0.2-10 second input to the reaction chamber.

14. The film of claim 13 which comprises the surface of a stent, catheter, implantable guidewire, implantable medical device, orthopedic device, pouch or mesh sack.

15. An artificial skin comprising an amorphous, nonconformal, porous titania or alumina coating 40-60 nm thick prepared by the method of claim 1 on a physiologically compatible dissolvable substrate.

16. The amorphous titania film of claim 13 which attracts any one or more of a cell selected from the group consisting of osteoblast, endothelial, gingival fibroblast and periodontal fibroblast cells.

17. An amorphous, thin porous titania film prepared by the method of claim 1 on a stainless steel substrate which attaches osteoblast, endothelial, gingival fibroblast and peridontal fibroblast cells compared to said surface on a polycarbonate substrate.
